(19) United States
(12) Patent Application Publication
(10) Pub. No.: US 2012/0035260 A1
(43) Pub. Date: Feb. 9, 2012

(54) TELLURIUM-CONTAINING COMPOUNDS FOR AFFECTING FEMALE'S REPRODUCTIVE SYSTEM FOLLOWING CHEMOTHERAPY AND/OR RADIOTHERAPY

(75) Inventors: Benjamin Sredni, Kfar-Saba (IL); Dror Meirow, Jerusalem (IL); Michael Albeck, Ramat-Gan (IL); Benjamin Ron, Savyon (IL)

(73) Assignee: BIOMAS LTD., Kfar-Saba (IL)

(21) Appl. No.: 13/143,544

(22) PCT Filed: Jan. 7, 2010
(86) PCT No.: PCT/IL10/00020

§ 371 Date: Oct. 4, 2011

Related U.S. Application Data
(60) Provisional application No. 61/193,917, filed on Jan. 8, 2009.
(60) Provisional application No. 61/213,655, filed on Jun. 30, 2009.

(51) Int. Cl. A61K 31/21; A61P 15/18; C07C 69/74

(52) U.S. Cl. 514/511; 560/116

(57) ABSTRACT

Use of tellurium-containing compounds in a method of conception and/or for maintaining and/or augmenting fertility in female following chemotherapy and/or radiotherapy is disclosed. The tellurium-containing compound is utilized in combination with a chemotherapeutic agent and/or radiation, such that the female treated by the chemotherapeutic agent and/or radiation and by the tellurium-containing compound is instructed to refrain from conceptive sex for a pre-determined time period, following the chemotherapy and/or radiotherapy, during which conception is undesired. The tellurium-containing compound is utilized in combination with a chemotherapeutic agent and/or radiation, such that the female treated by the chemotherapeutic agent and/or radiation and by the tellurium-containing compound is instructed to refrain from sex and/or from reproduction for a pre-determined time period, and is then allowed to practice reproduction.

### FIELD AND BACKGROUND OF THE INVENTION

The present invention, in some embodiments thereof, relates to a method of maintaining and/or augmenting fertility and, more particularly, but not exclusively, to a method of maintaining and/or augmenting fertility in a female subject undergoing chemotherapy and/or radiotherapy.

From the time of birth, the ovaries of a female comprise immature, primordial follicles, each follicle containing an immature oocyte. When a female reaches sexual maturity, a portion of the primordial follicles begin to develop. A primordial follicle matures over the course of approximately a year, commonly at least half a year, during which time the follicle gradually grows larger. During this period, the vast majority of the developing follicles die in a process termed “atresia”, until a single preovulatory follicle remains for each menstrual cycle. The oocyte of the preovulatory follicle then resembles meiosis and is secreted from the follicle during ovulation. This process continues until the primordial follicles have been depleted, at which point menopause occurs.

Chemotherapy, as well as radiotherapy directed to ovaries, have been shown to cause significant damage, including mutations, to oocytes at various stages of maturation. Exposure to chemotherapy during oocyte maturation has been found in animal studies to cause increases in abortions, fetal malformations and aneuploidy. Hence, there is major concern regarding the health of children conceived after the mother has received chemotherapy or radiotherapy. It has been recommended that women refrain from conceiving a child, or collecting oocytes for in vitro fertilization, until 6-12 months after treatment due to possible toxicity towards growing oocytes [Meirow & Schiff, J Natl Cancer Inst Monogr 2005; 34:21-25; Meirow et al., Hum Reprod 2001; 16:632-637].

In addition, damage to the ovaries caused by chemotherapy or radiotherapy may lead to sterilization or early menopause, by depleting the reserve of primordial follicles in the ovaries [Meirow & Nugent, Hum Reprod Update 2001; 7:535-543].

The effect of chemotherapy on the ovaries depends on the type of drug, dose, and schedule of treatment. Chemotherapeutic agents belonging to the class commonly referred to as “alkylating agents” (e.g., cyclophosphamide) are particularly toxic to gametes, and can cause both genetic damage to oocytes and ovarian failure [Arnon et al., Hum Reprod Update 2001; 7:394-403; Meirow & Nugent, Hum Reprod Update 2001; 7:535-543]. Alkylating agents are commonly used in the treatment of cancer as well as for other diseases, such as autoimmune diseases. Chemotherapeutic agents that are toxic to female's reproductive system are commonly referred to as gonadotoxic agents.

Tellurium-containing compounds have been shown in both preclinical and clinical studies to have beneficial effects against diverse complications caused by chemotherapeutic agents. Thus, the tellurium-containing compound AS101 was shown to protect mice from hematopoietic damage caused by lethal and sublethal doses of chemotherapeutic drugs, including cyclophosphamide (Cy), and to increase the survival of mice treated with various cytotoxic drugs or radiation, without negatively affecting treatment efficacy [Kalechman et al., Cancer Res 1991; 51:1499-1503; Kalechman et al., Radiat Res 1993; 136:197-204; Kalechman et al., Cancer Res 1993; 53:1838-1844; Kalechman et al., Immunopharmacology 1995; 29:149-158; Sredni et al., Int J Cancer 1996; 65:97-103; Sredni et al., Cancer Res 2004; 64:1843-1852].

Clinical trials in cancer patients treated with AS101 in combination with chemotherapy showed that treatment with AS101 induced a significant reduction in the severity of neutropenia, thrombocytopenia, and alopecia that accompany chemotherapy (Kalechman et al., Exp Hematol 1995; 23:1358-1366; Sredni et al., J Clin Oncol 1995; 13:2432-2353; Sredni et al., Int J Cancer 1996;65:97-103; Sredni et al., Cancer Res 2004; 64:1843-1852].

In addition, AS101 itself exhibits an anti-tumoral effect in a variety of tumor models in mice and humans. AS101 was found to have a synergistic effect with Cy in the treatment of tumor-bearing mice, suggesting that the combination of AS101 and Cy provides a more effective treatment of their tumors [Kalechman et al., Cancer Res 1991; 51:1499-1503].

AS101 sensitizes tumors to chemotherapy by inhibiting the tumor interleukin 10 autocrine loop, which results in decreased Stat3 activity, and by down regulation of the Akt/Survivin pathway [Kalechman et al., Int J Cancer 2000; 86:281-288; Sredni et al., FASEB J 2004; 18:400-402; Hayun et al., Biochem Pharmacol 2006; 72:1423-1431].

AS101 and other tellurium-containing compounds have been described in U.S. Pat. Nos. 4,752,614; 4,761,490; 4,764,461 and 4,929,739

Another family of bis-tellurium-containing compounds has been taught in WO 2006/030437.

### SUMMARY OF THE INVENTION

The present inventors have now surprisingly uncovered that tellurium-containing compounds such as AS101 prevent ovarian damage caused by chemotherapeutic agents and hence that (i) female patients undergoing chemotherapy should not assume fertility loss during and after chemotherapy, and thus should refrain from conceptive sex; and (ii) female patients can practice reproduction over a broader period of time following chemotherapy.

According to an aspect of some embodiments of the present invention there is provided a method of conception following chemotherapy and/or radiotherapy, the method comprising:

(a) administering to a female subject a therapeutically effective amount of a chemotherapeutic agent and/or radiation;

(b) administering to the female subject a gonadal-protective amount of a tellurium-containing compound;

(c) instructing the female to refrain from reproduction or refrain from sex for a predetermined time period following administration of the chemotherapeutic agent and/or radiation; and

(d) allowing the female to practice reproduction.

According to an aspect of some embodiments of the present invention there is provided a use of a tellurium-containing compound in the manufacture of a medicament for use in a method of conception following chemotherapy and/or radiotherapy, the medicament being for use in combination with a chemotherapeutic agent and/or radiation such that a female subject treated with the chemotherapeutic agent and/or radiation and with the tellurium-containing compound is instructed to refrain from reproduction or refrain from sex for a predetermined time period following administration of the chemotherapeutic agent and/or radiation, and is allowed to practice reproduction at the end of the pre-determined time period.

According to an aspect of some embodiments of the present invention there is provided a tellurium-containing compound being identified for use in a method of conception following chemotherapy and/or radiotherapy, the tellurium-containing compound being for use in combination with a chemotherapeutic agent and/or radiation such that a female subject treated with the chemotherapeutic agent and/or radiation and with the tellurium-containing compound is instructed to refrain from reproduction or refrain from sex for a predetermined time period following administration of the chemotherapeutic agent and/or radiation, and is allowed to practice reproduction at the end of the pre-determined time period.

According to an aspect of some embodiments of the present invention there is provided a pharmaceutical composition comprising a tellurium-containing compound and a pharmaceutically acceptable carrier, the composition being identified for use in a method of conception following chemotherapy and/or radiotherapy, in combination with a chemotherapeutic agent and/or radiation, such that a female subject treated with the chemotherapeutic agent and/or radiation and with the tellurium-containing compound is instructed to refrain from reproduction or refrain from sex for a predetermined time period following administration of the chemotherapeutic agent and/or radiation, and is allowed to practice reproduction at the end of the pre-determined time period.

According to some embodiments of the invention, the pharmaceutical composition is packaged in a packaging material and identified in print, in or on the packaging material, for use in the method of conception described herein.

According to some embodiments of the invention, the pharmaceutical composition further comprises the chemotherapeutic agent.

In some embodiments, reproduction is effected by conceptive sex with a potential father male.

In some embodiments reproduction is an assisted reproduction such as artificial insemination or in vitro fertilization.

According to some embodiments, the pre-determined time period is at least 12 months.

According to some embodiments, the pre-determined time period is at least 10 months.

According to some embodiments, the pre-determined time period is at least 8 months.

According to some embodiments, the pre-determined time period is at least 6 months.

According to some embodiments, the pre-determined time period is at least 5 months.

According to some embodiments, the pre-determined time period is at least 4 months.

According to some embodiments, the pre-determined time period is at least 3 months.

According to some embodiments, the pre-determined time period is at least 2 months.

According to some embodiments, the pre-determined time period is at least 1 month.

According to an aspect of some embodiments of the present invention there is provided a method of maintaining and/or augmenting female fertility following chemotherapy and/or radiotherapy, the method comprising:

(a) administering to a female subject a therapeutically effective amount of a chemotherapeutic agent and/or radiation;

(b) administering to the female subject a gonadal-protective amount of a tellurium-containing compound; and

(c) instructing the female to refrain from conceptive sex or refrain from sex for a predetermined time period.

According to an aspect of some embodiments of the present invention there is provided a use of a tellurium-containing compound in the manufacture of a medicament for maintaining and/or augmenting fertility in a female subject undergoing chemotherapy and/or radiotherapy, the medicament being for use in combination with a chemotherapeutic agent and/or radiation such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

According to an aspect of some embodiments of the present invention there is provided a tellurium-containing compound identified for use in maintaining and/or augmenting fertility in a female subject undergoing chemotherapy and/or radiotherapy, the tellurium-containing compound being for use in combination with a chemotherapeutic agent and/or radiation such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

According to an aspect of some embodiments of the present invention there is provided a pharmaceutical composition comprising a tellurium-containing compound and a pharmaceutically acceptable carrier, the composition being identified for use in maintaining and/or augmenting fertility in a female subject undergoing chemotherapy and/or radiotherapy, the tellurium-containing compound being for use in combination with a chemotherapeutic agent and/or radiation such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

According to some embodiments of the invention, the pharmaceutical composition is packaged in a packaging material and identified in print, in or on the packaging material, for use in combination with the chemotherapeutic agent and/or radiation, for maintaining and/or augmenting fertility in a female subject undergoing chemotherapy and/or radiotherapy, such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

According to some embodiments of the invention, the pharmaceutical composition further comprises the chemotherapeutic agent.

According to some embodiments, the pre-determined time period is less than 12 months.

According to some embodiments, the pre-determined time period is less than 10 months.

According to some embodiments, the pre-determined time period is less than 8 months.

According to some embodiments, the pre-determined time period is less than 6 months.

According to some embodiments, the pre-determined time period is less than 5 months.

According to some embodiments, the pre-determined time period is less than 4 months.

According to some embodiments, the pre-determined time period is less than 3 months.

According to some embodiments, the pre-determined time period is less than 2 months.

According to some embodiments, the pre-determined time period is less than 1 month.

According to some embodiments, the pre-determined time period is such that a level of serum anti-Mullerian hormone (AMH) of the female subject at the end of the time period is at least close to a normal or reference value.

According to some embodiments, any of the methods described herein further comprises, prior to administering to the female subject the chemotherapeutic agent and/or radiation:

determining a level of ovarian reserve serum Anti-Mullerian Hormone (AMH) in the female subject, the value being the reference value.

According to some embodiments, any of the methods described herein further comprises, subsequent to instructing the female subject to refrain from reproduction or conceptive sex or to refrain from sex:

determining a value of the level of serum anti-Mullerian hormone (AMH) in the female subject; and

determining if the value of a level of serum anti-Mullerian hormone (AMH) in the female subject is at least close to the reference value.

According to some embodiments, any of the uses, compositions and tellurium-containing compounds described herein are such that a level of ovarian reserve serum Anti-Mullerian Hormone (AMH) in the female subject is determined prior to administering to the female subject the chemotherapeutic agent and/or radiation, the value being the reference value.

According to some embodiments, any of the uses, compositions and tellurium-containing compounds described herein are such that subsequent to instructing the female subject to refrain from reproduction or conceptive sex or to refrain from sex, a value of the level of serum anti-Mullerian hormone (AMH) in the female subject is determined, and whether the value of a level of serum anti-Mullerian hormone (AMH) in the female subject is at least close to the reference value is also determined.

According to some embodiments, the tellurium-containing compound comprises at least one tellurium dioxo moiety.

According to some embodiments, the tellurium-containing compound has a general formula selected from the group consisting of:

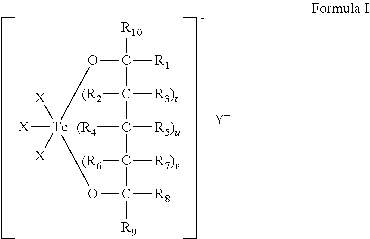

a compound having general Formula II:

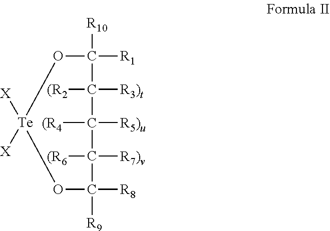

a compound having general Formula III:

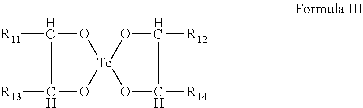

and
a compound having general Formula IV:

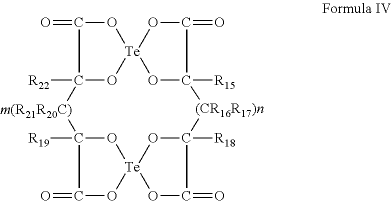

wherein:

each of t, u and v is independently 0 or 1;

each of m and n is independently 0, 1, 2 or 3;

Y is selected from the group consisting of ammonium, phosphonium, potassium, sodium and lithium;

X is a halogen atom; and

each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamide.

According to some embodiments, the tellurium-containing compound has the general Formula I.

According to some embodiments, t, u and v are each 0.

According to some embodiments, each of R₁, R₈, R₉ and R₁₀ is hydrogen.

According to some embodiments, X is chloro.

According to some embodiments, Y is ammonium.

According to some embodiments, the tellurium-containing compound is ammonium trichloro(dioxyethylene-O,O′)tellurate (AS101).

According to some embodiments, the compound has the general Formula IV.

According to some embodiments, each of m and n is 0.

According to some embodiments, each of R₁₅, R₁₈, R₁₉ and R₂₂ is hydrogen.

According to some embodiments, the tellurium-containing compound is SAS.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing the amount of primordial follicles (PMF) in female mice treated with 75, 100 or 150 mg/kg cyclophosphamide (Cy) or with PBS, with and without co-treatment with AS101;

FIG. 2 is a graph showing the amount of primordial follicles (PMF) in female mice treated 4 times with 75 mg/kg cyclophosphamide (Cy) or with PBS, with and without co-treatment with AS101;

FIGS. 3A and 3B are graphs showing the ratio of the populations of primary follicles (FIG. 3B) and secondary follicles (FIG. 3B) to the population of primordial follicles (PMF) in mice treated once with 75, 100 or 150 mg/kg cyclophosphamide (Cy) or 4 times with 75 mg/kg cyclophosphamide, with and without co-treatment with AS101;

FIG. 4 is a graph showing levels of anti-Mullerian hormone (AMH) in female mice treated 4 times with 75 mg/kg cyclophosphamide (Cy) or with PBS, with and without co-treatment with AS101; and

FIGS. 5A-5C are photographs of a growing follicle (FIG. 5A), showing apoptotic staining (pink) of granulosa cells treated with cyclophosphamide, of primordial follicles from ovary treated with AS 101 (FIG. 5B), showing normal primordial follicles, and of ovary exposed to chemotherapy (e.g., cyclophosphamide) and AS101 (FIG. 5C) showing follicles destroyed by chemotherapy positive apoptotic staining as well as healthy primordial follicles.

### DESCRIPTION OF SPECIFIC EMBODIMENTS OF THE INVENTION

The present invention, in some embodiments thereof, relates to a method of maintaining and/or augmenting fertility and, more particularly, but not exclusively, to a method of conception and/or of augmenting fertility in a female subject undergoing chemotherapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have surprisingly uncovered that tellurium-containing compounds may substantially reduce and even prevent ovarian damage caused by chemotherapeutic agents and radiation, thereby preserving ovarian function and protecting oocytes from damage caused by the chemotherapeutic agents and/or radiation. This phenomenon has opened the way for novel and advantageous methods for conception following chemotherapy and/or radiotherapy, for maintaining and augmenting fertility in females, and for reducing a risk of genetic defects in their offspring.

Thus, according to an aspect of some embodiments of the present invention, there is provided a method of conception following chemotherapy and/or radiotherapy, the method being effected by administering to a female subject a therapeutically effective amount of a chemotherapeutic agent and/or radiation; administering to the subject a gonadal-protective amount of a tellurium-containing compound; instructing the female subject to refrain from reproduction or refrain from sex for a predetermined time period following administration of the chemotherapeutic agent and/or radiation; and allowing the female to practice reproduction.

As used herein, the phrase “female subject” refers to an adult female, that is, a female old enough to be biologically capable of conceiving and giving birth to offspring.

As used herein, the phrase “method of conception following chemotherapy and/or radiotherapy” describes a method which enables a female subject to safely and/or effectively conceive a child after undergoing chemotherapy and/or radiotherapy.

By “safely” conceiving, it is meant conceiving with reduced risk to offspring (e.g., by reducing a risk of genetic defects in the oocyte and/or exposure of the embryo to chemotherapeutic agents) and/or to the female subject (e.g., by avoiding pregnancy during a period of time when the subject is not well enough to undergo pregnancy and childbirth).

By “effectively” conceiving, it is meant that the subject is capable of becoming pregnant and giving birth to a healthy child, and that loss of fertility is prevented, minimized and/or delayed.

In some embodiments, the method facilitates safe conception by avoiding conception (e.g., during the pre-determined time period following treatment) when a risk to the offspring and/or mother is relatively high.

In some embodiments, the method facilitates effective conception by preventing, minimizing and/or delaying a loss of fertility, such that the subject is fertile at the end of the predetermined time period. Optionally, the loss of fertility comprises premature menopause and/or ovarian failure, and the method of conception described herein prevents premature menopause and/or ovarian failure, or at least delays the premature menopause and/or ovarian failure.

According to some embodiments, the method facilitates both safe conception by avoiding conception during the predetermined time period, and facilitates conception after the end of the predetermined time period by preventing, minimizing and/or delaying a loss of fertility.

As used herein, the terms “chemotherapy” and “chemotherapeutic” refer to treatment with a chemical agent capable of causing damage (e.g., cell death and/or DNA mutation) to proliferating cells, typically cancer cells. The chemotherapy may be a treatment for a malignant disease or disorder (e.g., cancer), but chemotherapy for other conditions (e.g., autoimmune diseases, or conditions that require bone marrow ablation) is also intended. In some embodiments, these terms refer to treatment with chemotherapeutic agents that cause damage to gonadal tissue (e.g., gonads and/or follicles) and/or oocytes, either as an adverse side effect or per se. Such chemotherapeutic agents are referred to as gonadotoxic agents.

Chemotherapeutic agents suitable for use in embodiments of the present invention, include, without limitation, alkylating agents, vinca alkaloids (e.g., vincristine, vinblastine), antimetabolites (e.g., methotrexate, aminopterin, 5-fluorouracil, cytarabine), topoisomerase interactive agents (e.g., bleomycin, actinomycin, doxorubicin, daunorubicin), paclitaxel and other taxanes, and radiotherapeutic agents (e.g., radio-labeled antibodies).

In some embodiments, the chemotherapeutic agent is an alkylating agent, a platin derivate (e.g., cisplatin, carboplatin), a taxane, a vinca alkaloid, an antimetabolite, a topoisomerase interactive agent, or a radiotherapeutic agent, which is a gonadotoxic agent.

According to some embodiments of the present invention, the chemotherapeutic agent is an alkylating agent. Exemplary alkylating agents include, without limitation, nitrogen mustards (e.g., cyclophosphamide, mechlorethamine, uramustine, melphalan, chlorambucil, ifosfamide), nitrosoureas (e.g., carmustine, streptozocin), alkyl sulfonates (e.g., busulfan), thiotepa, platinum-based chemotherapeutic agents (e.g., cisplatin, carboplatin, nedaplatin, oxaliplatin, satraplatin, triplatin), procarbazine, altretamine, dacarbazine, mitozolomide and temozolomide.

Exemplary gonadotoxic alkylating agents include cyclophosphamide.

As used herein, the terms “radiation” and “radiotherapy” describe any external or internal radiation applied to a tissue to be treated (e.g., for cancer) to which gonadal tissue (e.g., gonads and/or follicles) and/or oocytes are at least somewhat exposed. In some embodiments, external radiation is applied to the ovaries or the surrounding area, for example, for treatment of a ovarian cancer or cervical cancer. In some embodiments, a radioactive agent (e.g., radio-labeled antibody) is administered systemically to the subject.

As used herein, the phrase “gonadal-protective amount” describes an amount sufficient to result in protection of gonads, follicles and/or oocytes against a damage caused by the chemotherapeutic agent and/or radiation. The protection may be in the form of preventing damage or reducing the degree of damage. In some embodiments, the chemotherapeutic agent and/or radiation causes transient or permanent cessation of ovulation (e.g., amenorrhea), and administration of a gonadal-protective amount of a tellurium-containing compound restores at least some of the ovulation. In some embodiments, the chemotherapeutic agent and/or radiation increases an amount of oocytes having genetic defects, and administration of a gonadal-protective amount of a tellurium-containing compound prevents or at least partially reverses the increase in oocytes with genetic defects. In some embodiments, the chemotherapeutic agent and/or radiation cause damage to the gonads, follicles and/or oocytes, and administration of a gonadal-protective amount of a tellurium-containing compound prevents or at least reduces this damage.

As used herein, the term “conception” refers to the act of becoming pregnant, by any form of reproduction.

As used herein, the term “reproduction” refers to a process of generating offspring. Reproduction can be effected by conceptive sex or by assisted reproduction.

As used herein, the phrase “conceptive sex” refers to any form of sexual intercourse (e.g., sexual intercourse without use of contraception) which may result in conception of a child. In some embodiments, a female can practice conceptive sex with a male who is a potential father (e.g., a fertile male).

The phrase “assisted reproduction” encompasses any reproductive technique that involves artificial or partially artificial means, including those that involve a third party.

Assisted reproduction, as used herein, therefore encompasses any technique by which the process of sexual intercourse is bypassed either by insemination or fertilization of the oocytes in the laboratory environment (in vitro fertilization (IVF).

In vitro fertilization (IVF) is a technique that involves fertilization of the male and female gametes (sperm and egg) which occurs outside the female body.

In vitro fertilization (IVF) can involve one or more of the following procedures: transvaginal ovum retrieval (OCR); assisted zona hatching (AZH); intracytoplasmic sperm injection (ICSI); autologous endometrial coculture; in zygote intrafallopian transfer (ZIFT); cytoplasmic transfer; and a gestational carrier, as these procedures are described in the art.

Additional assisted reproduction techniques include, but are not limited to, in gamete intrafallopian transfer (GIFT); Artificial insemination (AI); Use of conception devices, such as a conception cap; artificial insemination by donor; surrogacy; reproductive surgery; and in surgical sperm retrieval (SSR).

The duration of the predetermined time period during which the female subject is instructed, according to embodiments of the present invention, to refrain from reproduction (by e.g., conceptive sex or by assisted reproduction, will be determined by one of skill in the medical arts based on one or more of relevant factors including, without limitation, the dose, regimen and/or type of chemotherapeutic agent and/or radiation, the risk of genetic defects occurring in any offspring conceived as a result of the dose, regimen and/or type of the chemotherapeutic agent and/or radiation, the risk that the female subject will become infertile (e.g., by premature menopause and/or ovarian failure) by the end of, or soon after the end of, the predetermined time period, the dangers posed by chemotherapeutic agent in the body of the female subject to the conceived embryo, and the ability of the subject undergoing chemotherapy to withstand the exertion of pregnancy.

In some embodiments, the risk that the female subject will become infertile by the end of, or soon after the end of, the predetermined time period is a factor which at least partially determines the duration of the predetermined time period. It is to be appreciated that in such embodiments, the predetermined time period may optionally be longer than if a tellurium-containing compound is not administered, thereby reducing a risk to the offspring and/or female subject, because the compound prevents, minimizes and/or delays loss of fertility, as exemplified in the Examples section that follows, thereby extending the period of time during which the subject is biologically capable of conceiving a child (e.g., practice reproduction).

The skilled practitioner will appreciate that the risk that the female subject will become infertile by the end of, or soon after the end of, the predetermined time period depends on the subject's age. For example, premature menopause is more likely to occur in a female nearing an age at which menopause naturally occurs (e.g., female 35-45 years old) than in a younger female at an age far from that at which menopause naturally occurs (e.g., female 20-25 years old).

Thus, in some embodiments, the predetermined time period is at least 1 month, optionally at least 2 months, optionally at least 3 months, optionally at least 4 months, optionally at least 5 months, optionally at least 6 months, optionally at least 8 months, optionally at least 10 months, and optionally at least 12 months. Optionally, a short (e.g., about 1 month) predetermined time period is used, which reduces damage caused by, for example, exposure of the embryo and/or mature oocyte to a chemotherapeutic agent. Alternatively, a longer (e.g., about 6-12 months) predetermined time period is used, which further reduces damage caused by exposure of oocytes at most or all stages of maturation.

In some embodiments, the pre-determined time period is 6-12 months, which is the time period recommended to refrain from reproduction following chemotherapy and/or radiotherapy, when there is no known risk of infertility.

Typically, the predetermined time period will not be considerably more than 12 months, as no ovarian follicles exposed to chemotherapy and/or radiotherapy will remain after considerably more than 12 months.

In another aspect of embodiments of the present invention there is provided a use of a tellurium-containing compound in the manufacture of a medicament for use in a method of conception following chemotherapy and/or radiotherapy, the medicament being for use in combination with a chemotherapeutic agent and/or radiation such that a female subject treated with the chemotherapeutic agent and/or radiation and with the tellurium-containing compound is instructed to refrain from reproduction or refrain from sex for a predetermined time period following administration of the chemotherapeutic agent and/or radiation, and is allowed to practice reproduction at the end of the pre-determined time period.

In another aspect of some embodiments of the present invention there is provided a tellurium-containing compound being identified for use in a method of conception following chemotherapy and/or radiotherapy, the tellurium-containing compound being for use in combination with a chemotherapeutic agent and/or radiation such that a female ‘subject treated with the chemotherapeutic agent and/or radiation and with the tellurium-containing compound is instructed to refrain from reproduction or refrain from sex for a predetermined time period following administration of the chemotherapeutic agent and/or radiation, and is allowed to practice reproduction at the end of the pre-determined time period.

In another aspect of some embodiments of the present invention there is provided a pharmaceutical composition, as described herein, which comprises a tellurium-containing compound and a pharmaceutically acceptable carrier, the composition being identified for use in a method of conception following chemotherapy and/or radiotherapy, in combination with a chemotherapeutic agent and/or radiation such that a female subject treated with the chemotherapeutic agent and/or radiation and with the tellurium-containing compound is instructed to refrain from reproduction or refrain from sex for a predetermined time period following administration of the chemotherapeutic agent and/or radiation, and is allowed to practice reproduction at the end of the pre-determined time period.

In some embodiments, the pharmaceutical composition is packaged in a packaging material and identified in print, in or on the packaging material, for use in the method of conception, as described herein.

In some embodiments, the pharmaceutical composition further comprises the chemotherapeutic agent.

The tellurium-containing compound and the chemotherapeutic agent can be packaged together, in a single unit dosage form (e.g., co-formulation), or can be packaged separately in the packaging material.

In another aspect of embodiments of the present invention, there is provided a method of maintaining and/or augmenting female fertility following chemotherapy and/or radiotherapy, the method being effected by administering to a female subject a therapeutically effective amount of a chemotherapeutic agent and/or radiation; administering to the female subject a gonadal-protective amount of a tellurium-containing compound; and instructing the female to refrain from conceptive sex or refrain from sex for a predetermined time period following administration of the chemotherapeutic agent and/or radiation.

As used herein, the phrase “maintaining and/or augmenting fertility” describes preventing or reducing the degree of a loss of fertility of the female subject caused by a chemotherapeutic agent. The term “maintaining” herein means preventing a complete loss of fertility, such that at least some degree of fertility remains. The term “augmenting” herein means that a degree of fertility is caused to be higher than would be otherwise (e.g., a partial loss of fertility is prevented or reduced in degree). In some, but not all, embodiments, augmenting fertility comprises restoring a normal level of fertility. The fertility loss may be temporary or permanent. The fertility loss may represent a reduction in the ability to conceive offspring and/or a reduction in the likelihood that the conceived offspring will be healthy (e.g., free from genetic defects). In some embodiments, the loss of fertility is a reduction in the ability to conceive offspring which is a result of a choice made as a result of undergoing therapy to refrain from conceiving offspring, for example, in order to avoid the risk of conceiving offspring with genetic defects.

The duration of the predetermined time period during which the female subject is instructed, according to embodiments of the present invention, to refrain from conceptive sex will be determined by one of skill in the medical arts based on one or more of relevant factors, as described hereinabove.

In some embodiments, the risk of genetic defects occurring in offspring is a factor which at least partially determines the duration of the predetermined time period. It is to be appreciated that in such embodiments, the predetermined time period may optionally end sooner than if a tellurium-containing compound is not administered, because the compound reduces or eliminates damage to the oocytes, thereby reducing a risk of oocyte DNA damage, and thereby speeding recovery of normal, undamaged oocytes following chemotherapy and/or radiotherapy, or even maintaining normal, undamaged oocytes during the entire period following chemotherapy and/or radiotherapy.

Thus, in some embodiments, the predetermined time period is less than 12 months, optionally less than 10 months, optionally less than 8 months, optionally less than 6 months, optionally less than 5 months, optionally less than 4 months, optionally less than 3 months, optionally less than 2 months, and optionally less than 1 month.

As discussed hereinabove, the fertility loss in female subjects results from the effect of chemotherapy and/or radiation on various processes associated with fertility. The chemotherapy and/or radiation effect on these processes is reflected by a change in several parameters of a female subject undergoing chemotherapy. These include changes in ovarian function (e.g., ovulation, menstrual cycles, hormone levels), size of the primordial follicle reserve, and oocyte functionality (e.g., DNA structure of oocytes).

According to some embodiments of the present invention, the predetermined time period is determined such that gonads, follicles and/or oocytes of the female subject will not be considerably damaged at the end of the time period in comparison with normal gonads, follicles and/or oocytes, respectively.

According to some embodiments of the present invention, the predetermined time period is determined such that oocytes of the female subject will not be considerably damaged at the end of the time period in comparison with normal oocytes.

As exemplified herein in the Examples section, levels (e.g., serum levels) of anti-Mullerian hormone (AMH) may optionally serve as an indicator of ovarian function.

Hence, in some embodiments, damage to ovaries and/or oocytes is determined by determining a level (e.g., serum level) of AMH, such that the oocytes are not considered considerably damaged if a level of AMH is at least close to normal.

Herein, the term “normal” describes the expected level in the female subject had the subject not undergone chemotherapy or radiotherapy.

In some embodiments, the method is further effected by determining a value of a level of AMH in the subject prior to administering the chemotherapeutic agent and/or radiation, and using the obtained value as a reference value to define normal ovarian and/or oocyte function.

Alternatively, reference values may optionally be obtained by other means, for example, based on average values reported in the medical literature.

As used herein, the phrase “at least close to normal” means ±50% of the normal value, optionally ±20%, and optionally ±10%.

According to some embodiments, the method is further effected by determining values of an AMH level of the female subject subsequent to instructing the female subject to refrain from conceptive sex, and determining if the value of the AMH level is at least close to the reference value.

Optionally, the aforementioned determining of a value of AMH levels is performed at or near the end of the predetermined time period in order to confirm that the value has returned to, or is at least close to the reference value, such that the subject may safely practice conceptive sex.

Optionally, if the value at the end of the predetermined time period is not at least close to the reference value, the predetermined time period is followed by a second predetermined time period during which the subject is further instructed to refrain from practicing conceptive sex.

It is noted that the method, according to this aspect of embodiments of the invention, prevents a risk of conception (e.g., upon practicing conceptive sex, as defined herein) during the time period following chemotherapy and/or radiotherapy in which damage to ovaries and/or oocytes is likely to occur, whereby such a risk results from an assumption that the female's fertility has been lost during treatment and hence that a conception cannot be practiced. As described herein, subjecting the female subject to a co-treatment with a tellurium-containing compound prevents fertility loss, and hence enhances the risk of conception during a time period in which conception is undesired.

Further according to some embodiments of the invention, there is provided a use of a tellurium-containing compound in the manufacture of a medicament for maintaining and/or augmenting fertility in a female subject undergoing chemotherapy and/or radiotherapy, the medicament being for use in combination with a chemotherapeutic agent and/or radiation such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

Further according to some embodiments of the invention, there is provided a tellurium-containing compound identified for use in maintaining and/or augmenting fertility in a female subject undergoing chemotherapy and/or radiotherapy, the tellurium-containing compound being for use in combination with a chemotherapeutic agent and/or radiation such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

Further according to some embodiments of the invention, there is provided a pharmaceutical composition comprising a tellurium-containing compound and a pharmaceutically acceptable carrier, the composition being identified for use in maintaining and/or augmenting fertility in a female subject undergoing chemotherapy and/or radiotherapy, the tellurium-containing compound being for use in combination with a chemotherapeutic agent and/or radiation such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

In some embodiments of the invention, the pharmaceutical composition is packaged in a packaging material and identified in print, in or on the packaging material, for use in combination with the chemotherapeutic agent and/or radiation, for maintaining and/or augmenting fertility in a female subject undergoing chemotherapy and/or radiotherapy, such that the female subject receiving the chemotherapeutic agent and/or radiation and the tellurium-containing compound is instructed to refrain from conceptive sex or refrain from sex for a predetermined time period following administration of the chemotherapeutic agent and/or radiation.

In some embodiments of the invention, the pharmaceutical composition further comprises the chemotherapeutic agent, as described herein.

In some embodiments of the invention, in any of the aspects of the invention described herein, determining a value of a level of AMH in the subject prior to administering the chemotherapeutic agent and/or radiation, and using the obtained value as a reference value to define normal ovarian and/or oocyte function, as described herein, is effected.

Accordingly, determining values of an AMH level of the female subject subsequent to instructing the female subject to refrain from reproduction, and determining if the value of the AMH level is at least close to the reference value, as described herein, is effected. Optionally, the aforementioned determining of a value of AMH levels is performed at or near the end of the predetermined time period in order to confirm that the value has returned to, or is at least close to the reference value, such that the subject may safely practice reproduction.

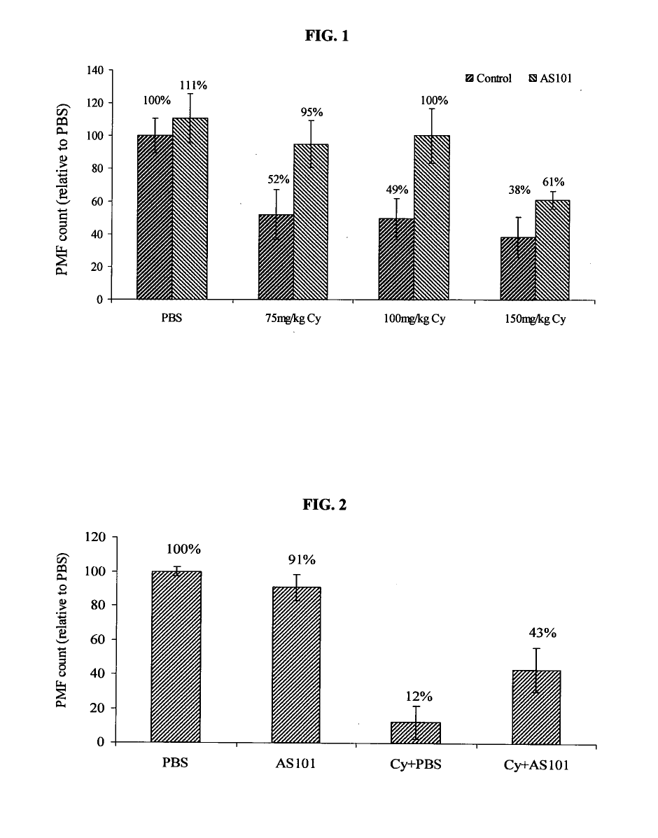

Referring now to the drawings, FIGS. 1, 2, 3A and 3B show that cyclophosphamide depletes primordial follicles and increases ratios of growing follicles to primordial follicles, and that this phenomenon is reversed by administration of a tellurium-containing compound.

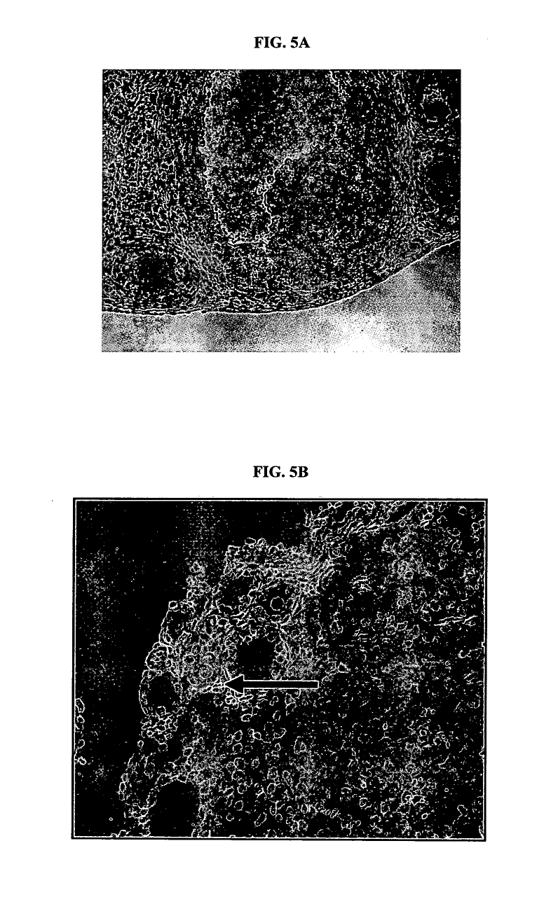

FIG. 4 shows that administration of a tellurium-containing compound protects against cyclophosphamide-induced reduction in AMH levels.

Without being bound to any particular theory, it is believed that the experimental results described herein indicate that reduction in AMH levels represents a reliable indicator of damage induced by chemotherapy and/or radiotherapy to ovaries and/or oocytes.

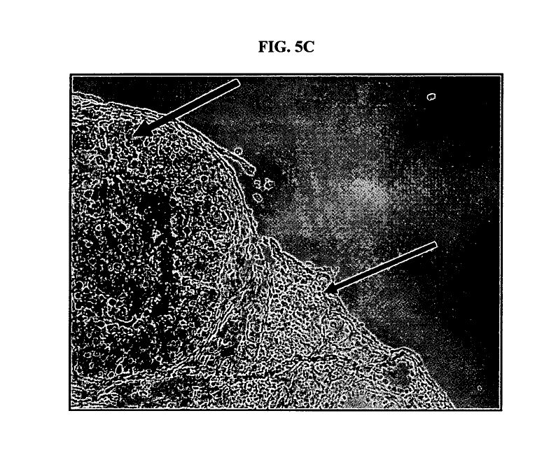

FIGS. 5A-5C show cyclophosphamide-induced apoptosis in growing follicles (FIG. 5A), normal primordial follicles from ovary treated with AS101 (FIG. 5B), and follicles destroyed by chemotherapy as well as healthy primordial follicles of ovary exposed to chemotherapy (e.g., cyclophosphamide) and AS101 (FIG. 5C).

In any of the methods, compositions and uses described herein, a tellurium-containing compound, which comprises one or more tellurium atoms, is utilized.

In some embodiments, the tellurium-containing compound comprises at least one tellurium dioxo moiety.

Herein throughout, the phrases “tellurium dioxo moiety” and “tellurium dioxide moiety” are used interchangeably, and describe an —O—Te—O—, in which the tellurium center can be further substituted, or a O═Te═O.

The tellurium-containing compound may be an inorganic compound or an organic compound.

Inorganic tellurium-containing compounds include, for example, tellurium dioxide (TeO₂) per se.

Organic tellurium-containing compounds may be in the form of an organic complex such as, for example, a TeO₂ complex with citric acid or ethylene glycol, which may form TeO₂ as an end product in aqueous solutions. A representative example of the latter is the complex TeO₂.HOCH₂CH₂OH.NH₄Cl. Otherwise, the tellurium-containing compounds described herein include one or more tellurium atoms and one or more organic moieties that are attached thereto, for example, ammonium salts, or any other salts, of halogenated tellurium-containing compounds having a bidentate cyclic moiety attached to the tellurium atom.

Exemplary compounds in this category can be collectively represented by the general Formula I:

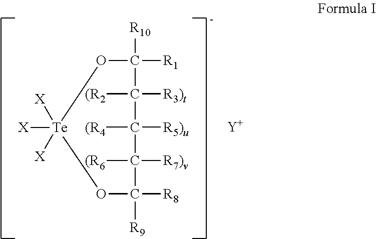

In the general Formula I above, each of t, u and v is independently 0 or 1, such that the compound may include a five-membered ring, a six-membered ring, or a seven-membered ring. In some embodiments, each of t, u and v is 0, such that the compound includes a five-membered ring.

X is a halogen atom, as described hereinabove, and is preferably chloro.

Y can be ammonium, phosphonium, potassium, sodium and lithium, and is preferably ammonium.

Each of R₁-R₁₀ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

As used herein, the term “alkyl” refers to a saturated aliphatic hydrocarbon including straight chain and branched chain groups. In some embodiments, the alkyl group has 1 to 20 carbon atoms. In some embodiments, the alkyl is a medium size alkyl having 1 to 10 carbon atoms. In some embodiments, the alkyl is a lower alkyl having 1 to 5 carbon atoms. The alkyl group may be substituted or unsubstituted. When substituted, the substituent group can be as described herein for R₁.

As used herein, the term “hydroxyalkyl” refers to an alkyl, as this term is defined herein, substituted by a hydroxy group, as defined herein, and includes, for example, hydroxymethyl, hydroxyethyl, hydroxypropyl and hydroxy-n-butyl.

As used herein, the term “halogen”, which is also referred to herein interchangeably as “a halogen atom” or “halo”, includes chloro (Cl), bromo (Br), iodo (I) and fluoro (F).

The term “haloalkyl” refers to an alkyl, as this term is defined herein, substituted by a halogen, as defined herein, and includes, for example, chloromethyl, 2-iodoethyl, 4-bromo-n-butyl, iodoethyl, 4-bromo-n-pentyl and the like.

The term “alkanoyloxy” refers to a carbonyl group, as define herein and includes, for example, acetyl, propionyl, butanoyl and the like.

The term “carboxyalkyl” refers to an alkyl, as this term is defined herein, substituted by a carboxy group, as defined herein, and includes, for example, carboxymethyl, carboxyethyl, ethylenecarboxy and the like.

The term “alkylcarbonylalkyl” refers to an alkyl, as this term is defined herein, substituted by a carbonyl group, as defined herein, and includes, for example, methanoylmethyl, ethanoylethyl and the like.

The term “amidoalkyl” refers to an alkyl, as this term is defined herein, substituted by an amide group, as defined herein, and includes, for example, —CH₂CONH₂; —CH₂CH₂CONH₂; —CH₂CH₂CH₂CONH₂ and the like.

The term “cyanoalkyl” refers to an alkyl, as this term is defined herein, substituted by an cyano group, as defined herein, and includes, for example, —CH₂CN; —CH₂CH₂CN; —CH₂CH₂CH₂CN and the like.

The term “N-monoalkylamidoalkyl” refers to an alkyl, as this term is defined herein, substituted by an amide group, as defined herein, in which one of R′ and R″ is an alkyl, and includes, for example, —CH₂CH₂CONHCH₃, and —CH—₂CONHCH₂CH₃.

The term N,N-dialkylamidoalkyl refers to an alkyl, as this term is defined herein, substituted by an amide group, as defined herein, in which both R′ and R″ are alkyl, and includes, for example, —CH₂CON(CH₃)₂; CH₂CH₂CON(CH₂—CH₃)₂ and the like.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be as described herein for R1.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be as described herein for R1.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. When substituted, the substituent group can be as described herein for R1.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. Examples, without limitation, include, piperazine, piperidine, morpholine, tetrahydrofuran and tetrahydropyran. The heteroalicyclic may be substituted or unsubstituted. When substituted, the substituent group can be as described herein for R1.

A “hydroxy” group refers to an —OH group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein.

A “carboxy” group refers to a —C(═O)—O—R′ or a —O—C(═O)—R′ group, where R′ is as defined herein.

A “sulfinyl” group refers to an —S(═O)—R′ group, where R′ is as defined herein.

A “sulfonyl” group refers to an —S(═O)₂—R′ group, where R′ is as defined herein.

A “sulfate” group refers to a —O—S(═O)₂—OR′ group, where R′ is as defined herein.

A “sulfonamido” group refers to a —S(═O)₂—NR′R″ group or a R′S(═O)₂—NR″, with R′ is as defined herein and R″ is as defined for R′.

A “carbamyl” or “carbamate” group refers to an —OC(═O)—NR′R″ group or a R″OC(═O)—NR′— group, where R′ and R″ are as defined herein.

A “thiocarbamyl” or “thiocarbamate” group refers to an —OC(═S)—NR′R″ group or an R″OC(═S)NR′— group, where R′ and R″ are as defined herein.

An “amino” group refers to an —NR′R″ group where R′ and R″ are as defined herein.

An “amido” group refers to a —C(═O)—NR′R″ group or a R′C(═O)—NR″ group, where R′ and R″ are as defined herein.

A “nitro” group refers to an —NO₂ group.

A “cyano” group refers to a group.

The term “phosphonyl” describes a —O—P(═O)(OR')(OR″) group, with R′ and R″ as defined hereinabove.

The term “phosphinyl” describes a —PR′R″ group, with R′ and R″ as defined hereinabove.

As cited hereinabove, the compounds in this category are salts of organic tellurium-containing compounds. The salts can be, for example, ammonium salts, phosphonium salts and alkaline salts such as potassium salts, sodium salts, lithium salts and the like.

Hence, Y in Formula I above can be a phosphonium group, as defined herein, an ammonium group, as defined herein, potassium (K⁺), sodium (Na⁺) or lithium (Li⁺).

As used herein, the term “phosphonium” describes a —P⁺R′R″R′″ group, with R′ and R″ as defined herein and R′″ is as defined for R′. The term “phosphonium”, as used herein, further refers to a —P⁺R₆ group, wherein each of the six R substituents is independently as defined herein for R, R″ and R′″.

The term “ammonium” describes a —N⁺R′R″R′″ group, with R′, R″ and R′″ as defined herein.

In some embodiments, compounds in this category include compounds having the general Formula I described above, in which Y is ammonium or phosphonium, t, u and v are each 0, and each of R₁, R₈, R₉ and R₁₀ is independently hydrogen or alkyl. These compounds can be represented by the following structure:

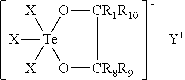

wherein each of R₁, R₈, R₉ and R₁₀ is independently hydrogen or alkyl, whereas, in some embodiment, the alkyl is methyl, and X is halogen, preferably chloro.

In some embodiments, a tellurium-containing compound for use in the context of the present embodiments has the following structure:

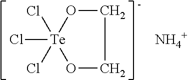

This compound is ammonium trichloro(dioxyethylene-O,O′)tellurate, which is also referred to herein and in the art as AS101.

Additional representative examples of organic tellurium-containing compound that are suitable for use in the context of the present invention include halogenated tellurium having a bidentate cyclic moiety attached to the tellurium atom. The bidentate cyclic moiety is preferably a dioxo ligand having two oxygen atoms attached to the tellurium atom.

Exemplary compounds in this category can be represented by the general Formula II:

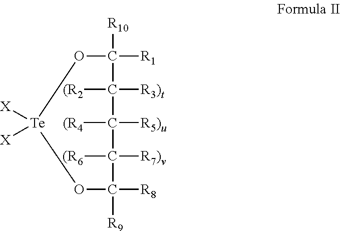

wherein t, u, v, X and R₁-R₁₀ are as defined hereinabove.

In some embodiments, the tellurium-containing compounds are those in which t, u, and v are each 0, and X is chloro, such as, but not limited to, the compound having the following structure:

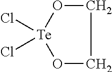

The above compound is also known in the art and referred to herein as AS103.

The organic tellurium-containing compounds having Formulae I and II can be readily prepared by reacting tetrahalotelluride such as TeCl₄ with a dihydroxy compound, as is described in detail in U.S. Pat. Nos. 4,752,614, 4,761,490, 4,764,461 and 4,929,739, which are incorporated by reference as if fully set forth herein.

Additional representative examples of organic tellurium-containing compounds that are suitable for use in the context of the present embodiments include compounds in which two bidentatic cyclic moieties are attached to the tellurium atom. Preferably, each of the cyclic moieties is a dioxo moiety.

Exemplary compounds in this category are collectively represented by the general Formula III:

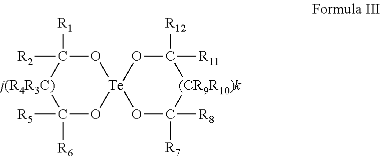

In the general Formula III above, each of j and k is independently an integer from 0 to 4, such that the compound may include a five-membered ring, a six-membered ring, a seven-membered ring, an eight-membered ring and/or a nine-membered ring. In some embodiments, each of j and k is an integer from 0 to 2, such that the compound includes a five-membered ring, a six-membered ring and/or a seven-membered ring. In some embodiments, each of j and k is 0.

R₁-R₁₂ are as defined hereinabove for R₁-R₁₀.

In some embodiments, tellurium-containing compounds in this category are those in which j and k are each 0, and R₃, R₄, R₉ and R₁₀ are each hydrogen, having the following structure:

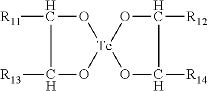

wherein each of R₁₁-R₁₄ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido, as these terms are defined herein.

In some embodiments, a tellurium-containing compound in this category is a compound in which each of R₁₁-R₁₄ is hydrogen. This compound is also known in the art and referred to herein as AS102.

Additional representative examples of organic tellurium-containing compounds that are suitable for use in the context of the present embodiments include the recently disclosed ditellurium compounds having general Formula IV:

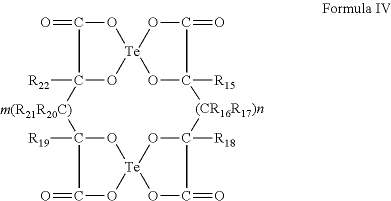

wherein each of R₁₅-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamide, as these terms are defined herein; and

m and n are each an integer from 0 to 3.

Exemplary compounds in this category are those in which m and n are each 0.

An exemplary compound in this family is a compound in which R₁₅, R₁₈, R₁₉ and R₂₂ are all hydrogen, referred to hereinafter as SAS, and which has the following structure:

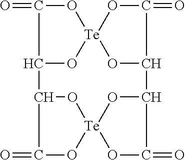

According to some embodiments of the present invention, the tellurium-containing compound is either AS101 or SAS, as described herein.

The compounds described above can be administered or otherwise utilized in the various aspects of the present invention, either as is or as a pharmaceutically acceptable salt thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

The tellurium-containing compound and the chemotherapeutic agent (and/or radiation), utilized in embodiments of the methods and uses described herein, may be administered concomitantly. Alternatively, the tellurium-containing compound may be administered before or after the chemotherapeutic agent and/or radiation (i.e., sequentially).

In any of the methods and uses described herein, administration of the tellurium-containing compound and optionally of additional active agents (e.g., the chemotherapeutic agent) can be performed via various routes of administrations.

Suitable routes of administration may, for example, include the inhalation, oral, buccal, rectal, transmucosal, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a tissue region of a subject.

Determination of a gonadal-protective amount of a tellurium-containing compound is well within the capability of those skilled in the art.

For any preparation used in the methods of the invention, the gonadal-protective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity of the potential damage caused by the chemotherapy and/or radiotherapy, dosing can be of a single or a plurality of administrations.

When administering systemically, a therapeutically effective amount of the tellurium-containing compounds described herein may range, for example, from about 0.01 mg/m²/day to about 20 mg/m²/day and thus can be for example, 0.01 mg/m²/day, 0.02 mg/m²/day, 0.03 mg/m²/day, 0.04 mg/m²/day, 0.05 mg/m²/day, 0.1 mg/m²/day, 0.5 mg/m²/day, 1 mg/m²/day, 2 mg/m²/day, 3 mg/m²/day, 4 mg/m²/day, 5 mg/m²/day, and up to 10 mg/m²/day. Preferably, for systemic administration, a gonadal-protective amount of a compound of formula I, II, III or IV ranges from about 0.01 mg/m²/day to about 10 mg/m²/day. Higher gonadal-protective amounts, such as, for example, up to 20 mg/m²/day can also be employed.

In one embodiment, when administered intraperitoneally, the gonadal-protective amount is 0.01 mg/m²/day and higher and thus can be, for example,0.01 mg/m²/day, 0.05 mg/m²/day, 0.1 mg/m²/day, 0.2 mg/m²/day, 0.5 mg/m²/day, 0.6 mg/m²/day, 0.7 mg/m²/day, 0.8 mg/m²/day, 0.9 mg/m²/day, 1 mg/m²/day, 2 mg/m²/day, 3 mg/m²/day, 4 mg/m²/day, 5 mg/m²/day, and up to 20.0 mg/m²/day. When administered orally in humans, a daily dose typically ranges between 0.1 mg and 200 mg, more preferably between 1 mg and 100 mg, depending on the age and weight of the subject. The total daily dose may be administered as a single dosage, or may be divided into a number of separate doses.

In any of the methods and uses described herein, the tellurium-containing compound and the chemotherapeutic agent can form a part of a pharmaceutical composition (either each alone or in combination), which further comprises a pharmaceutically acceptable carrier. Pharmaceutical compositions comprising one or more tellurium-containing compound described herein may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with embodiments of the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients may be formulated in aqueous solutions, preferably in physiologically compatible buffers such as Hank's solution, Ringer's solution, or physiological salt buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise glass, plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In one embodiment, the pharmaceutical composition described herein is packaged in a packaging material and identified in print, in or on said packaging material, for use in the maintenance and/or augmentation of fertility of a female subject undergoing chemotherapy and/or radiotherapy, as described herein.

In another embodiment, the pharmaceutical composition is identified for use in a method of conception following chemotherapy and/or radiotherapy, as described herein.

Optionally, the pharmaceutical composition is further identified for use in combination with the chemotherapeutic agent used for chemotherapy and/or the radiation.

In one embodiment, a concentration of tellurium-containing compound in the carrier ranges from about 0.01 weight percent to about 50 weight percents, more preferably from about 0.1 weight percent to about 25 weight percents, of the total weight of the composition.

It is expected that during the life of a patent maturing from this application many relevant chemotherapeutic agents will be developed and the scope of the term “chemotherapeutic agent” is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Material and Methods

Experimental Design:

6 week old female Balb/c mice were divided randomly into 4 groups. The mice in the treatment groups received cyclophosphamide (Cy) with or without co-treatment with AS101. Control mice received injections of phosphate buffer saline (PBS) or AS101. Cy treatment consisted of a single intraperitoneal injection of 75, 100 or 150 mg/kg Cy, or 4 weekly injections of 75 mg/kg Cy. AS101 was injected at a dose of 10 mg/kg every other day starting 1 week prior to Cy administration until 1 week after the last administration of Cy. PBS was injected on the same days as described for AS101.

Determination of PMF Counts and Follicle Populations:

Both ovaries were removed 7 days after the last Cy administration treatment, and fixed in 4% paraformaldehyde in PBS. The ovaries were embedded in paraplast and serially sectioned to 5 μm slices, and stained with haematoxylin/eosin. Care was taken to ensure that both ovaries were removed from each mouse in their entirety for histological processing. Primordial, primary and secondary follicles were counted by two blinded examiners who were unaware of the treatments, using a light microscope (AxioImager Z1, Zeiss, Oberkochen, Germany) in bright-field mode using a X20 objective. To avoid counting the same follicle twice, only one section in each set of five consecutive sections was used. The relative distribution of different stages of early follicle growth was then calculated.

Follicles were classified as primordial follicles (PMF) if they contained an oocyte surrounded by a partial or complete layer of squamous pregranulosa cells without a theca layer. In this species, PMF are located almost exclusively in the ovarian cortex, and they are very small (about 15 μm diameter). The follicular count of each follicle type was then multiplied by 5 to reach a representative of total follicles [Meirow et al., Hum Reprod 1999; 14:1903-1907]. The data from follicular count is presented as the percentage of the count obtained from the PBS-treated control group.

Determination of Anti Mullerian Hormone Levels:

Anti-Mullerian hormone (AMH) levels in the plasma were measured by ELISA techniques according to the manufacturer instruction (DSL, Webster, Tex.).

TUNEL Staining for Apoptosis:

Follicular apoptosis was visualized using the TUNEL staining of ovaries from additional group of mice sacrificed at 12, 24 and 48 hours after a single injection of 150 mg/kg Cy.

Statistics:

Student's T-test was performed to assess the differences between groups. Statistical significance was determined at p<0.05.

### Results

PMF Counts:

As shown in FIG. 1, single injections of 75 mg/kg, 100 mg/kg, and 150 mg/kg of Cy reduced the ovarian PMF count to 52.1%±15.1%, 48%±12.6%, and 38%±22.2% of that of the control (PBS) group, respectively, whereas in the groups treated with Cy+AS101 the PMF count was 94.8%±14.4%, 99.8%±16.7%, and 60.7%±5.3% of that of the control group, respectively.

At all tested doses of Cy, the difference between the PMF count of the Cy group and the PMF of both the PBS and Cy+AS101 groups was statistically significant.

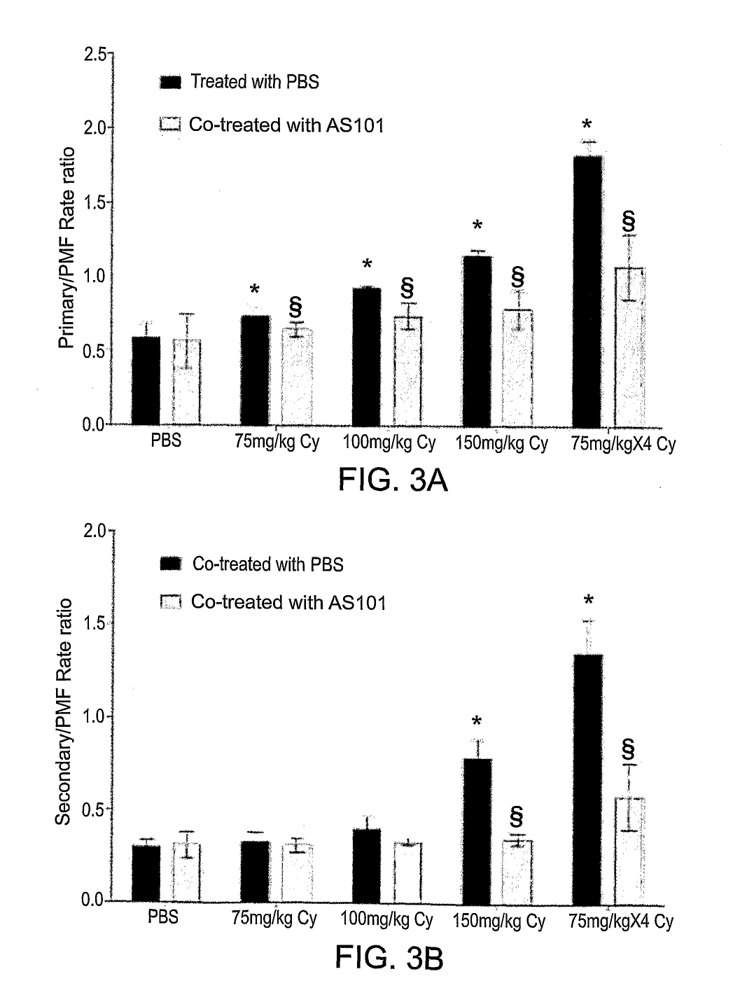

As shown in FIG. 2, administration of 75 mg/kg once a week for 4 weeks has reduced the PMF count to 12.2%±9.5% of that of the control group, whereas co-treatment with AS101 resulted in a PMF count of 43.0%±12.9% of that of the control group. The difference in PMF count between the Cy and Cy+AS101 groups was statistically significant.

### Population Ratios of Follicles:

The populations of primary follicles, secondary follicles and primordial follicles (PMF) were characterized following single injections of 75, 100 or 150 mg/kg Cy or 4 weekly injections of 75 mg/kg Cy.

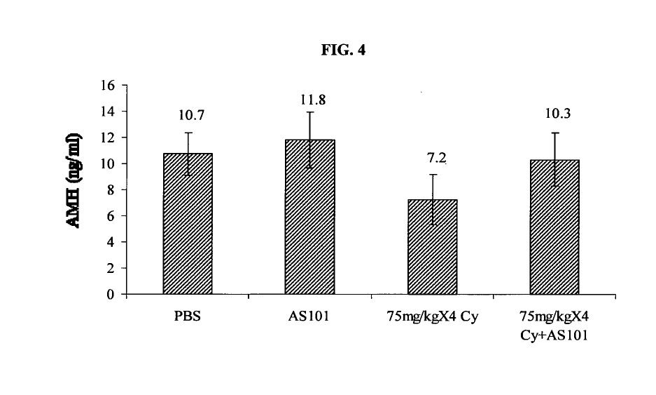

As shown in FIGS. 3A and 3B, Cy administration increased the ratio of both primary follicles (FIG. 3A) and secondary follicles (FIG. 3B) relative to primordial follicles in a dose dependent manner, whereas co-treatment with AS101 prevented the Cy-induced increase in these ratios.

AMH Levels:

Mice were administered PBS, AS101, Cy or Cy+AS101 for 4 weeks, as described hereinabove.

As shown in FIG. 4, Cy decreased the plasma AMH levels from 10.7±2.1 ng/ml to 7.2±1.9 ng/ml, whereas in the Cy+AS101 group, the AMH levels were 10.3±2.0 ng/ml. The AMH levels in the Cy+AS101 group were similar to those in the control (PBS) group (10.7±2.1 ng/ml), and represented a statistically significant increase relative to the Cy group.

TUNEL Staining:

As shown in FIG. 5A, extensive apoptotic staining was observed in the granulosa cells of growing follicles 24 hours after a single treatment with 150 mg/kg Cy.

In contrast, as shown in FIG. 5B, no apoptotic stain was detected in primordial follicles from ovary treated with AS101. As shown in FIG. 5C, upon co-therapy with a chemotherapeutic agent and AS101, follicles destroyed by chemotherapy as well as healthy primordial follicles of ovary are observed (see arrows).

There were no observed differences between results 12, 24 and 48 hours after Cy administration (results for 12 and 48 hours not shown).

The above results indicate that AS101 confers protection against Cy-induced damage to ovarian follicles.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-47. (canceled)

48. A method of conception following chemotherapy and/or radiotherapy, the method comprising:
(a) administering to a female subject a therapeutically effective amount of a chemotherapeutic agent and/or radiation;
(b) administering to said female subject a gonadal-protective amount of a tellurium-containing compound;
(c) instructing said female to refrain from reproduction or refrain from sex for a predetermined time period following administration of said chemotherapeutic agent and/or radiation; and
(d) allowing said female to practice reproduction.

49. A tellurium-containing compound being identified for use in a method of conception following chemotherapy and/or radiotherapy, the tellurium-containing compound being for use in combination with a chemotherapeutic agent and/or radiation such that a female subject treated with said chemotherapeutic agent and/or radiation and with said tellurium-containing compound is instructed to refrain from reproduction or refrain from sex for a predetermined time period following administration of said chemotherapeutic agent and/or radiation, and is allowed to practice reproduction at the end of said pre-determined time period.

50. A method of maintaining and/or augmenting female fertility following chemotherapy and/or radiotherapy, the method comprising:
(a) administering to a female subject a therapeutically effective amount of a chemotherapeutic agent and/or radiation;
(b) administering to said female subject a gonadal-protective amount of a tellurium-containing compound; and
(c) instructing said female to refrain from conceptive sex or refrain from sex for a predetermined time period.

51. A tellurium-containing compound identified for use in maintaining and/or augmenting fertility in a female subject undergoing chemotherapy and/or radiotherapy, the tellurium-containing compound being for use in combination with a chemotherapeutic agent and/or radiation such that said female subject receiving said chemotherapeutic agent and/or radiation and the tellurium-containing compound is instructed to refrain from conceptive sex or refrain from sex for a predetermined time period following administration of said chemotherapeutic agent and/or radiation.

52. The tellurium-containing compound of claim 51, further including a pharmaceutically acceptable carrier.

53. The method of claim 48, wherein said pre-determined time period is selected from the group consisting of at least 12 months, at least 6 months, at least 1 month and such that a level of serum anti-Mullerian hormone (AMH) of said female subject at the end of said time period is at least close to a normal or reference value.

54. The tellurium-containing compound of claim 49, wherein said pre-determined time period is such that a level of serum anti-Mullerian hormone (AMH) of said female subject at the end of said time period is at least close to a normal or reference value.

55. The method of claim 53, wherein said pre-determined time period is such that a level of serum anti-Mullerian hormone (AMH) of said female subject at the end of said time period is at least close to a normal or reference value and further comprising, prior to administering to said female subject said chemotherapeutic agent and/or radiation:
determining a level of ovarian reserve serum Anti-Mullerian Hormone (AMH) in said female subject, said value being said reference value.

56. The method of claim 55, further comprising, subsequent to instructing said female subject to refrain from conceptive sex or from reproduction or to refrain from sex:
determining a value of said level of serum anti-Mullerian hormone (AMH) in said female subject; and
determining if said value of a level of serum anti-Mullerian hormone (AMH) in said female subject is at least close to said reference value.

57. The tellurium-containing compound of claim 49, wherein said tellurium-containing compound comprises at least one tellurium dioxo moiety.

58. The tellurium-containing compound of claim 57, wherein said tellurium-containing compound has a general formula selected from the group consisting of: 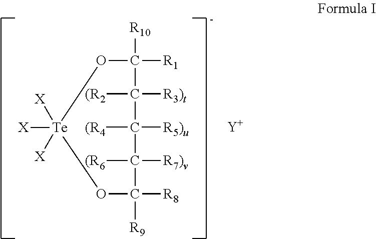
a compound having general Formula II: 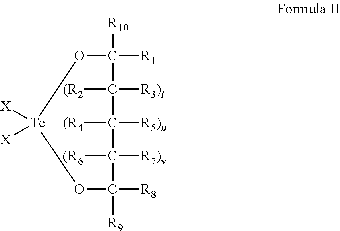
a compound having general Formula III: 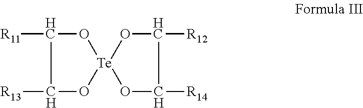
and
a compound having general Formula IV: 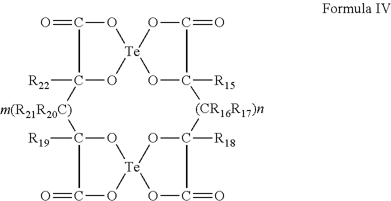
wherein:
each of t, u and v is independently 0 or 1;
each of m and n is independently 0, 1, 2 or 3;
Y is selected from the group consisting of ammonium, phosphonium, potassium, sodium and lithium;
X is a halogen atom; and
each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

59. The tellurium-containing compound of claim 58, wherein said tellurium-containing compound has said general Formula I.

60. The tellurium-containing compound of claim 59, wherein t, u and v are each 0.

61. The tellurium-containing compound of claim 59, wherein each of R₁, R₈, R₉ and R₁₀ is hydrogen.

62. The tellurium-containing compound of claim 58, wherein said tellurium-containing compound is ammonium trichloro(dioxyethylene-O,O′)tellurate (AS101).

63. The tellurium-containing compound of claim 58, wherein said compound has said general Formula IV.

64. The tellurium-containing compound of claim 63, wherein each of m and n is 0.

65. The tellurium-containing compound of claim 64, wherein each of R₁₅, R₁₈, R₁₉ and R₂₂ is hydrogen.

66. The tellurium-containing compound of claim 63, wherein said tellurium-containing compound is SAS.

67. The method of claim 48, wherein said tellurium-containing compound is one of ammonium trichloro(dioxyethylene-O,O′)tellurate (AS101) and SAS.
